(19) United States
(12) Patent Application Publication
(10) Pub. No.: US 2023/0417679 A1
(43) Pub. Date: Dec. 28, 2023

(54) Testing Device, System, and Methods for Detecting Contaminants

(71) Applicant: SAVR Pak Corporation, San Juan Capistrano, CA (US)

(72) Inventors: William Birgen, Tempe, AZ (US); Grant Stafford, San Juan Capistrano, CA (US)

(73) Assignee: SAVR Pak Corporation, San Juan Capistrano, CA (US)

(21) Appl. No.: 18/252,937

(22) PCT Filed: Nov. 17, 2021
(86) PCT No.: PCT/US2021/072452

§ 371 Date: May 15, 2023

Related U.S. Application Data
(60) Provisional application No. 63/114,650, filed on Nov. 17, 2020.

(51) Int. Cl. G01N 21/78; G01N 33/02

(52) U.S. Cl. CPC G01N 21/78; G01N 33/02

(57) ABSTRACT

A carrier with a reagent selected to detect a contaminant and provide an alert is provided on a media in the environment of a consumable containing the contaminant and at a temperature below the dew point of the environment, which causes condensation of the media and allows for detection and alert of the contaminant.

### FIELD OF THE INVENTION

The present invention relates to the field of storing and testing food or consumables for bacteria/bacterium, allergens, toxins, or other detectable contaminants

### BACKGROUND OF THE INVENTION

Food or medicine may become contaminated leading to illness and death. Moreover, it is difficult and costly to test food for bacteria/bacterium or contaminants which may arise through inadvertent exposure to contaminated food or food preparers or cross contamination at a large food processing center or kitchen. Similarly, large batches of produce may come from a farm where water and subsequently the food, has become contaminated inadvertently from waste created by adjacent independent animal farms. It would be advantageous to be able to provide an indication that a consumable has become contaminated with bacteria/bacterium, allergens, toxins, or other undesirable detectable contaminants.

### SUMMARY OF THE INVENTION

Aspects of the testing system and methods disclosed herein are implemented in devices or methods to detect presence at least one reagent-reactive compound such as a toxin, bacterium, carcinogen, allergen, etc., (hereinafter “contaminants”) using reagents that are configured to indicate of the presence of the reagent-reactive compound or contaminant. Aspects may be implemented by suspending the reagent in a carrier below the dew point of the environment in which the consumable is positioned and exposing the reagent to moisture from the environment in which the consumable is stored. Aspects of the invention may be implemented in a food or consumable storage and testing device that tests consumables without making physical contact with the consumable. Other aspects may be implemented in a device that may be attached to a food container to test a consumable in the food container without making physical contact between the device and the consumable. Still other aspects may be implemented in a process of using the device to test a consumable for a contaminant by including a reagent in or on a carrier at or below the dew point of the environment of the consumable to condense moisture from the environment and exposure or communication to the reagent. As further disclosed herein, the consumable testing device, system, or process may test a consumable with at least one reagent capable of detecting a contaminant transferred to the reagent, and which reagent then will react to the contaminant and manifest a user detectable indication of the presence of the at least one contaminant.

Aspects of disclosure include intentionally positioning a frozen reagent carrier with a reagent selected to react with a contaminant in the environment or direct vicinity of the consumable having the contaminant, which frozen reagent carrier will have a temperature below the dew point temperature of the environment or direct vicinity of the consumable and will thus draw, condense, or absorb moisture from the environment of the consumable and communicate or expose absorbed moisture from the environment to the at least one reagent selected to detect the contaminant. The reagent may provide a detectable reaction or alert, such as a visual alert, or the reagent may be combined with at least one sensor configured to detect the reagent reaction with the contaminant and communicate the presence of the contaminant. Aspects of the disclosure provide for the combination of a plurality of reagents selected to react with a plurality of contaminants, which may be combined with at least one sensor capable of communicating the presence or absence of at least one of the contaminants.

Aspects of the disclosure may be implemented in a method or device for testing food for contaminants such as bacteria/bacterium (e.g., E. coli), allergen(s) (e.g., peanuts), or other unwanted contaminants prior to consumption. One exemplary implementation comprises food container for premade single serving meals, food to go, food delivery, catering, room service, etc.

Aspects of the disclosure also comprises system of components working together to sample moisture from the air surrounding the food to be tested for at least one potentially hazardous contaminant.

Numerous advantages and features of the present invention will become readily apparent from the following detailed description of the invention and the embodiments thereof, from the claims and from the accompanying drawings.

### BRIEF DESCRIPTION OF THE DRAWINGS

FIG. 1 illustrates an embodiment of a foot testing embodiment or device 10 incorporating aspects of the invention;

FIGS. 2-3 illustrate examples of use of the device 10 in a hard sided and soft sided food container, respectively; and

FIGS. 4A-4B illustrate a device 10 wherein the reagent 30 reacts to produce a visible indicator 12 of the presence of a contaminant.

The objects, features and advantages of the present invention will be more readily appreciated upon reference to the following disclosure when considered in conjunction with the accompanying drawings, wherein reference numerals are used to identify the components in the various views.

### DESCRIPTION OF PREFERRED EMBODIMENT(S)

The figures illustrate a food testing device and system embodiment incorporating aspects of the invention. Aspects disclosed may be scaled or modified for all human or animal consumables and the accompanying storage, serving, or delivery systems. It is contemplated that one embodiment of the invention is a food or consumable storage and testing device and system that can detect bacteria/bacterium, allergens, toxins, or other undesirable detectable contaminants (hereinafter, “contaminants”) and communicate the presence of said contaminants in the consumable storage and testing device and system. The device embodiment may alert a user that the consumable in the consumable storage and testing device and system is either contaminated or not contaminated, and in alternate embodiment may alert as to a change in character, presence, or bacterial variety or population of a contaminant in the device. Further additional applications of the aspects disclosed herein may become obvious considering the disclosure herein. Accordingly, the features of the disclosed embodiment should not be construed as limiting any aspect of the invention.

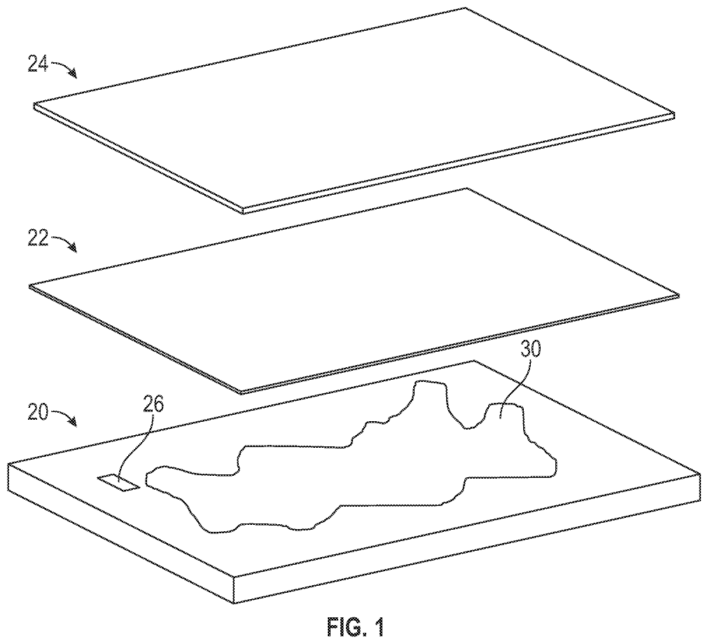

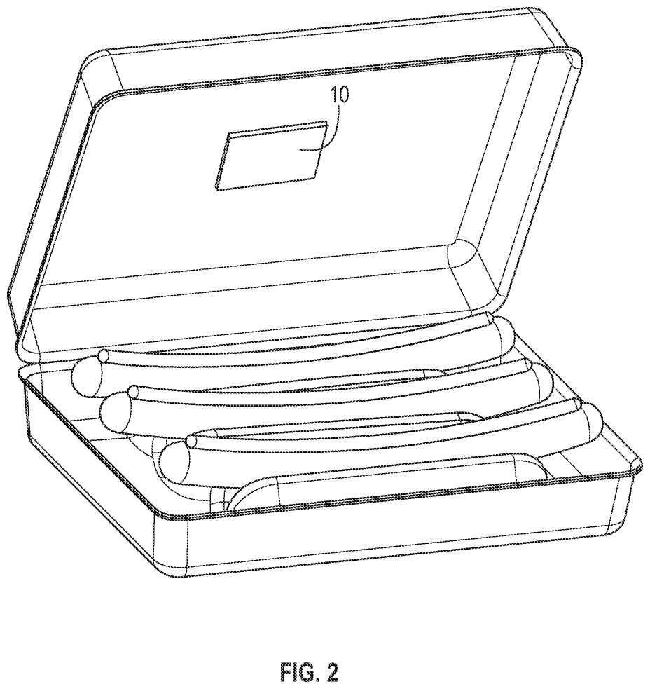

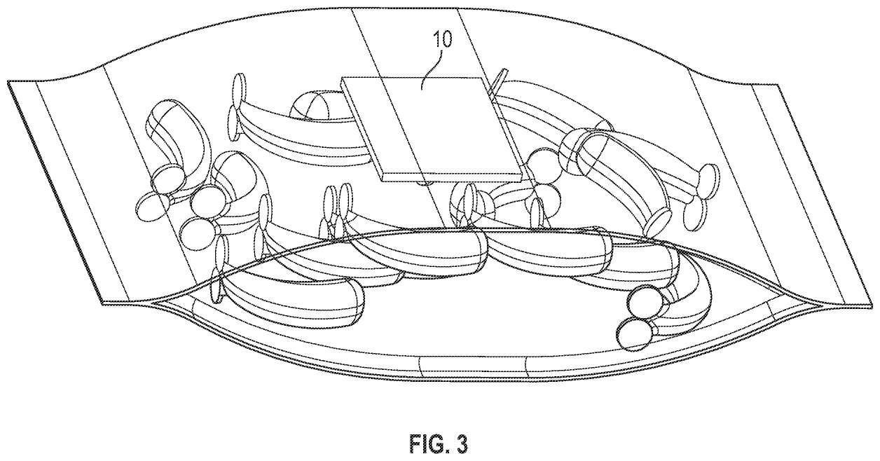

The figures show examples of installations of a testing device incorporating aspects of the invention and comprise use, positioning, or positioning of said devices in food containers, which could be containers with frozen food, quick serve food, or other food ready to eat, such as bagged or premixed salad from the supermarket, or food delivery, or takeaway food, or room service. These are just examples of typical uses or installs and should not be construed as limiting practical additional implementations. An exploded view of one embodiment invention is illustrated in FIG. 1. A surface of the absorbent media 20 may have an adhesive 22 is affixed to the inside top lid of any food container. A protective film 24 may be peeled away from the adhesive 22 prior to attaching the device to the inside of the container. Similarly, the adhesive 22 of the device may adhere to any surface inside a food container such as a bag, a box, a clamshell structure, a refrigerator's crisper drawer, etc.

The testing device and system embodiment illustrated is preferably designed, retrofitted, configured, or assembled to test a consumable within an environment. The environment may be preexisting or specifically created and considered as sufficient to create a distinct environment comprised of the vapor or gases emitted from the consumable, compared or relative to the vapor or gasses existing outside of the environment. As an example, a food storage or take-away container or a bagged prepared food (e.g., salad, greens, hemp, cannabis, etc.) that may be or is enclosed creates an environment inside the container or bag that is distinct as compared to the environment outside the container or bag. In one embodiment, a device for detecting a contaminant in the environment includes a reagent 30 selected to react to the contaminant and wherein the reagent 30 is selected or configured to provide an alert in the presence of the contaminant impregnated or stored on an absorbent media 20 and wherein the combination is provided in the environment at or below the dew point of the environment. In the preferred embodiment, the reagent 30 may be provided in or suspended in a liquid carrier 32 preferably comprised of or including water, and the combination of reagent 30 and liquid carrier 32 provided in or on the absorbent media 20. Under such conditions vapor or gasses emitted from the consumable inside the test environment will condense in or on the absorbent media 20 and allow functional communication with or exposure to the reagent 30 that is selected for its ability to react to the contaminant and provide an alert that is detectable to a user or other sensor capable of providing a visible, audible, or other electronic alert. In one implementation of the preferred embodiment, the reagent 30 and carrier 32 is frozen within the absorbent media 20 that may be selected from air laid, water-absorbing polymer, plant pulp, and hemp fiber, and an adhesive may be provided on a surface of the absorbent media 20 that may be adhered to an inside surface of a food container. Moreover, a protective, removable/disposable protective film may be applied over the adhesive and is peeled away to expose the adhesive prior to use. It is contemplated that the testing device is to be installed by the food preparer as the food is being packaged. If a reagent 30 reacts, manifesting a color, identifying an allergen or bacterium, it will be observed prior to food consumption by either a food handler, delivery person, delivery robot, or by the food consumer, or the consumer's caregiver. Identifying a reagent 30 reaction will potentially save the life of the would-be consumer of “tainted” food.

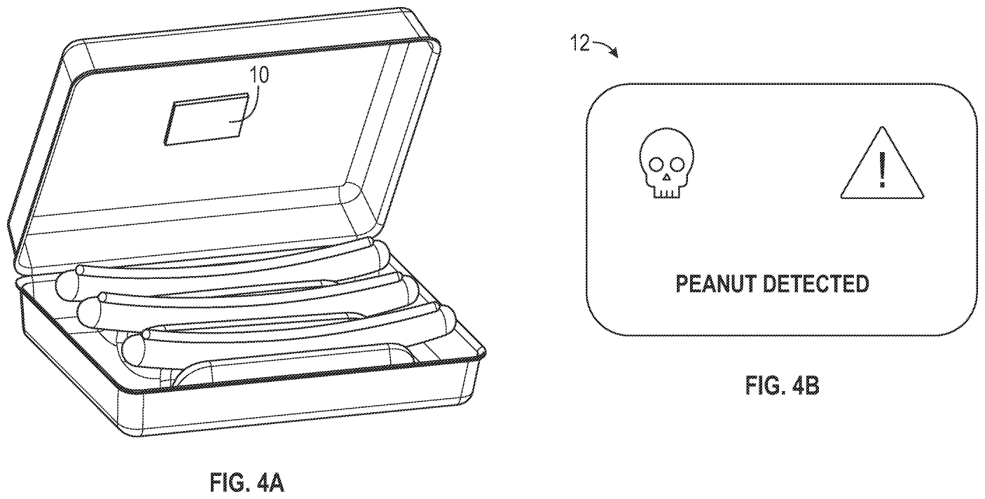

A reagent 30 is selected for its ability to withstand freezing temperatures and its ability to react to and provide a detectable alert. Exemplary reagents 30 include reagents selected for the ability to detect contaminants such as but not limited to foodborne pathogens and their toxins and surface spread contaminants, such as E. coli, Citrobacter freundii, Enterobacter aerogenes, copper sulphate, etc. One example of a reagent 30 comprises Colorless 3,5,30,50 tetramethyl benzidine (TMB), which changes to blue oxidized TMB in the presence of E. coli and a low concentration of H₂O₂ (e.g., ˜11 mM) at pH of 3. (See Rapid Detection of Pathogenic Bacteria by the Naked Eye, Kandasamy, Jannatin, and Yu-Chie Chen, Biosensors 2021, 11, 317. https://doi.org/10.3390/bios11090317). Notification that a potentially hazardous food or food contaminant has been discovered, presents itself through a color change occurring within the absorbent structure, i.e., reagent 30. Said absorbent media 20 is integrated with some nominal mass of ice, (e.g., 20-60 mL per single serving container) which, being below the dew point temperature, forcibly and quickly extracts moisture from the air. This air, having been in contact with the food is imparted with contaminants or food allergen(s) responsive to the selected reagent 30. Further, as illustrated in FIGS. 4A-4B, the presence of a detected contaminant may be communicated to the user or food service worker or delivery person by a reagent 30 configured in letters, words, icons, or symbols and that becomes visible or colored visible indicator on the surface of the absorbent media 20 in the presence of detected contaminant.

Additionally, a reagent sensor 26 as illustrated in FIG. 1 configured to detect the color change or other detectable reagent 30 reaction with the contaminant may be combined with the reagent 30 on the absorbent media 20 and the notification that a potentially hazardous food or food contaminant has be discovered, may present itself through a wireless communication system; e.g., RFID, or Bluetooth, Zigbee, etc. For example, a reagent 30 may be augmented by a reagent sensor 26 on the absorbent media 20 that detects the presence of ammonia in the instance of aging meat and when a threshold of contamination is detected/met an alert is transmitted or read wirelessly.

While various embodiments have been described above, they have been presented by way of example only, and not limitation. Thus, the breadth and scope of a preferred embodiment should not be limited by any of the above-described exemplary embodiments but should be defined only in accordance with the following claims and their equivalents.

1. A device for detecting a contaminant in an environment, comprising:
a reagent selected to react to the contaminant and configured to provide an alert upon reaction with the contaminant; and
an absorbent media including the reagent and a carrier containing water, the carrier capable of a temperature below the dew point of the environment, the absorbent media configured to absorb condensation from the environment;
wherein the device may be provided below the dew point of the environment to produce the alert in the presence of contaminant.

2. The device in claim 1 wherein,
the carrier is frozen.

3. The device in claim 1 wherein,
the absorbent media is selected from air laid, water-absorbing polymer, plant pulp, and hemp fiber.

4. The device in claim 1 further comprising,
an adhesive is provided on a first surface of the absorbent media, wherein the device is adhered to an inside surface of a food container.

5. The device in claim 4 wherein,
the alert comprises a visible indicator located at a second surface of the absorbent media.

6. The device in claim 5 wherein,
the visible indicator comprises a pattern selected from letters, words, colors, icons, and symbols in the presence of detected contaminant.

7. The device in claim 1 further comprising,
a sensor configured to detect the reagent reaction with the contaminant, the sensor configured for wireless transmission of detected presence of contaminant.

8. The device in claim 1 further comprising,
a tray upon which the absorbent media is positioned.

9. A device for detecting a contaminant, comprising:
a container capable of at least partial enclosure to create a test target environment;
an absorbent media positioned to functionally communicate with the test target environment; and
a reagent selected to react to the contaminant, positioned on the absorbent media;
wherein a test target with a contaminant may be placed in the container, the absorbent media may functionally communicate with the test target environment, and the reagent may react with the contaminant.

10. capable of being provided with a reagent temperature less than the test target

11. A method for detecting a contaminant, comprising:
creating an at least partially enclosure test environment;
positioning a reagent for exposure with the test environment, the reagent selected for its ability for a reaction to the contaminant;
condensing moisture from the test environment to expose the reagent to the moisture.
detecting the reaction to the contaminant; and
providing a first alert after detecting the reaction.

12. The method in claim 11 wherein,
the reagent 30 is provided in a frozen carrier that contains water.

13. The method in claim 11 wherein,
the reagent is provided on an absorbent media and condensing moisture comprises condensing moisture on the absorbent media.

14. The method in claim 13 wherein,
the absorbent media is selected from air laid, water-absorbing polymer, plant pulp, and hemp fiber.

15. The method in claim 13 further comprising,
detecting a non-reaction of the reagent to the contaminant, and
providing an alternate alert of the non-reaction to the contaminant.
